(19) United States
(12) Patent Application Publication
(10) Pub. No.: US 2026/0000292 A1
(43) Pub. Date: Jan. 1, 2026

(54) Method for Accurate Measurement of Macular Pigment Optical Density Using Autofluorescence Imaging with Melanin Consideration

(71) Applicant: Mohsen Sharifzadeh, Cottonwood Heights, UT (US)

(72) Inventor: Mohsen Sharifzadeh, Cottonwood Heights, UT (US)

(21) Appl. No.: 18/757,450

(22) Filed: Jun. 27, 2024

(51) Int. Cl. A61B 3/12; A61B 5/00; G06T 7/00

(52) U.S. Cl. CPC A61B 3/12; A61B 5/0071; G06T 7/0012; A61B 2576/02; G06T 2207/10064; G06T 2207/20224; G06T 2207/30041

(57) ABSTRACT

This invention introduces an improved method for measuring macular pigment optical density (MPOD) using autofluorescence imaging (AFI). Traditional AFI methods often neglect melanin absorption, leading to inaccuracies. The proposed method employs three wavelengths: 488 nm for the main image, 514 nm as a traditional baseline, and 600 nm as an additional baseline to account for melanin absorption. By converting the melanin absorption coefficient from 600 nm to 488 nm and 514 nm, this method ensures precise MPOD measurements. Additionally, a simplified dual-wavelength method is introduced using 488 nm and 600 nm for scenarios requiring less complexity. Both approaches enhance the accuracy of non-invasive retinal imaging by providing a more reliable tool for assessing macular pigment levels and improving our understanding of retinal health.

### BACKGROUND OF THE INVENTION

The current methods for measuring macular pigment optical density (MPOD) using autofluorescence imaging (AFI) often neglect the impact of melanin in the macula, leading to potential inaccuracies. Traditional AFI methods use two wavelengths, such as 488 nm and 514 nm, where lipofuscin absorbs the excitation light and generates autofluorescence. These methods involve comparing the main image (488 nm) and the baseline image (514 nm) to calculate MPOD by subtracting the baseline from the main image and applying compensation factors based on macular pigment absorption bands. However, these approaches do not account for the absorption by melanin, which can affect the accuracy of MPOD measurements.

Melanin is a significant pigment in the retinal pigment epithelium (RPE) and the macula, especially concentrated in the fovea. Its presence influences optical measurements due to its light absorption properties. Traditional methods fail to isolate the impact of melanin, leading to potential underestimation or overestimation of MPOD.

The proposed method addresses this limitation by introducing a second wavelength that is absorbed by lipofuscin but not by macular pigment, typically above 550 nm. This wavelength generates lipofuscin autofluorescence and provides a baseline image that includes the influence of melanin. By converting the melanin absorption coefficient from this second wavelength to the 488 nm wavelength, the new method accurately accounts for melanin's impact, ensuring precise MPOD measurements.

This advancement in AFI methodology enhances the accuracy of non-invasive retinal imaging, providing a more reliable tool for assessing macular pigment levels and improving our understanding of retinal health.

### BRIEF DESCRIPTION OF THE DRAWINGS

FIG. 1: Optical absorption spectra of macular pigment (MP) and lipofuscin. The absorption peaks around 488 nm and 532 nm are highlighted, demonstrating the overlap and the need for accurate correction for melanin absorption.

FIG. 2: Optical absorption spectrum of melanin showing its absorption across a wide range of wavelengths, emphasizing the importance of correcting for melanin absorption in MPOD measurements.

FIG. 3: Autofluorescence image of the macula generated using 488 nm excitation. The light absorbed by macular pigment and lipofuscin generates lipofuscin autofluorescence.

FIG. 4: Autofluorescence image of the macula generated using 514 nm excitation. This wavelength is also absorbed by macular pigment and lipofuscin, generating lipofuscin autofluorescence.

FIG. 5: Autofluorescence image of the macula generated using 600 nm excitation. The light is absorbed by lipofuscin but not by macular pigment, generating lipofuscin autofluorescence and accounting for melanin absorption.

FIG. 6: Graph showing macular pigment optical density (MPOD) and melanin optical density (OD) in one subject, demonstrating that melanin accounts for approximately 30% of the apparent MPOD. This underscores the importance of correcting for melanin absorption to obtain accurate MPOD measurements.

FIG. 7: Conversion of the melanin absorption coefficient from 600 nm to 488 nm and 514 nm. The absorption at 600 nm is first converted to the equivalent absorption at 488 nm using a compensation factor b488 and then converted to the equivalent absorption at 514 nm using a compensation factor b514. This process corrects for melanin absorption, ensuring accurate measurement of macular pigment optical density (MPOD).

FIG. 8: Calculation process for accurate macular pigment optical density (MPOD) measurement using the triple-wavelength approach. The primary AFI image generated at 488 nm is combined with the first baseline AFI image at 514 nm and the second baseline AFI image at 600 nm. The melanin absorption coefficients are converted and subtracted to obtain the corrected MPOD at 450 nm.

FIG. 9: Calculation process for accurate macular pigment optical density (MPOD) measurement using the dual-wavelength approach. The primary AFI image generated at 488 nm is combined with the baseline AFI image at 600 nm. The melanin absorption coefficient is converted and subtracted to obtain the corrected MPOD at 450 nm.

### DETAILED DESCRIPTION

### Method 1: Triple-Wavelength Approach

### Selection of Wavelengths

Primary Wavelength: 488 nm, absorbed by macular pigment (MP), which includes lutein, zeaxanthin, and meso-zeaxanthin, and lipofuscin, generating lipofuscin autofluorescence.
First Baseline Wavelength: 514 nm, also absorbed by MP and lipofuscin, generating lipofuscin autofluorescence.
Second Baseline Wavelength: 600 nm, absorbed by lipofuscin but not by MP, generating lipofuscin autofluorescence and accounting for melanin absorption.

### Generation of Primary and Baseline AFI Images

Barrier Filter: A barrier filter is used to block crystalline lens autofluorescence, ensuring accurate measurement of retinal autofluorescence.
Primary AFI Image: Generated using 488 nm, influenced by both MP and lipofuscin.
First Baseline AFI Image: Generated using 514 nm, influenced by both MP and lipofuscin.
Second Baseline AFI Image: Generated using 600 nm, influenced by melanin and lipofuscin.

### Calculation of MPOD Considering Melanin

1. Primary Measurement: Measure MPOD at 488 nm.
2. First Baseline Adjustment: Measure MPOD using 514 nm.
3. Second Baseline Adjustment: Measure melanin absorption using 600 nm.
4. Coefficient Conversion: Convert melanin absorption from 600 nm to 488 nm and 514 nm.
5. Corrected MPOD Calculation: Subtract the converted melanin values from the primary MPOD measurement at 488 nm and 514 nm to find the corrected MPOD at 450 nm.

### Mathematical Formulation

$${MPOD}_{450} = K . [ log {( \frac{{I}_{p e r}}{{I}_{m a c}} )}_{488} - a . log {( \frac{{I}_{p e r}}{{I}_{m a c}} )}_{514} - [ {b}_{488} . log {( \frac{{I}_{p e r}}{{I}_{m a c}} )}_{600} + {b}_{514} . log {( \frac{{I}_{p e r}}{{I}_{m a c}} )}_{600} ]$$

Where a is the compensation factor for the absorption coefficient difference between 488 nm and 514 nm, and b488 and b514 are the compensation factors for converting melanin absorption from 600 nm to 488 nm and 514 nm respectively.

### Method 2: Dual-Wavelength Approach

### Selection of Wavelengths

Barrier Filter: A barrier filter is used to block crystalline lens autofluorescence, ensuring accurate measurement of retinal autofluorescence.
Primary Wavelength: 488 nm, absorbed by MP (lutein, zeaxanthin, and meso-zeaxanthin) and lipofuscin, generating lipofuscin autofluorescence.
Baseline Wavelength: 600 nm, absorbed by lipofuscin but not by MP, generating lipofuscin autofluorescence and accounting for melanin absorption.

### Generation of Primary and Baseline AFI Images

Primary AFI Image: Generated using 488 nm, influenced by both MP and lipofuscin.
Baseline AFI Image: Generated using 600 nm, influenced by melanin and lipofuscin.

### Calculation of MPOD Considering Melanin

1. Primary Measurement: Measure MPOD at 488 nm.
2. Baseline Adjustment: Measure melanin absorption using 600 nm.
3. Coefficient Conversion: Convert melanin absorption from 600 nm to 488 nm.
4. Corrected MPOD Calculation: Subtract the converted melanin value from the primary MPOD measurement at 488 nm to find the corrected MPOD at 450 nm.

### Mathematical Formulation

$${MPOD}_{450} = K . [ log {( \frac{{I}_{p e r}}{{I}_{m a c}} )}_{488} ] - [ {b}_{488} . log {( \frac{{I}_{p e r}}{{I}_{m a c}} )}_{600} ]$$

Where K is a constant for the conversion to 450 nm, and b488 is the compensation factor for converting melanin absorption from 600 nm to 488 nm.

[symbol]

### FIGURE CAPTIONS

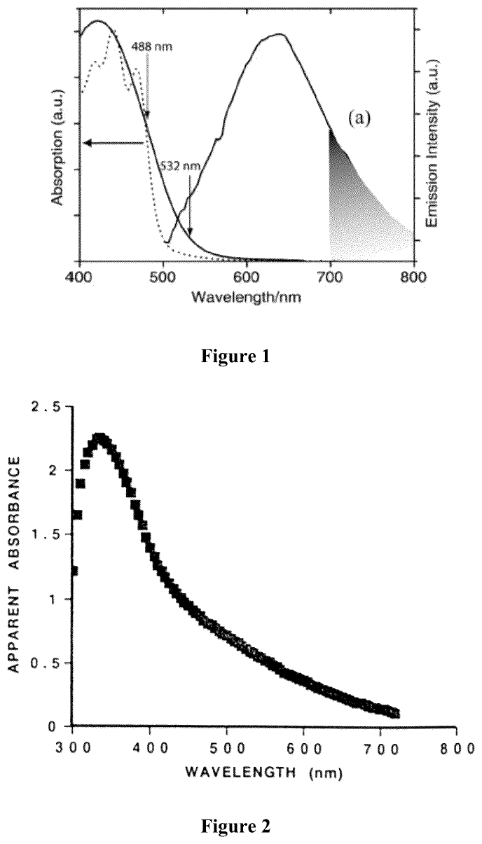

FIG. 1: Optical absorption spectra of macular pigment (MP) and lipofuscin. The absorption peaks around 488 nm and 532 nm are highlighted, demonstrating the overlap and the need for accurate correction for melanin absorption. (Source: [J Opt Soc Am A Opt Image Sci Vis. 2006 October; 23 (10): 2373-2387.])

FIG. 2: Optical absorption spectrum of melanin, showing its absorption across a wide range of wavelengths, emphasizing the importance of correcting for melanin absorption in MPOD measurements. (Source: [“The spectroscopy of human melanin pigmentation,” by N. Kollias. In: Melanin: Its Role in Human Photoprotection, pp. 31-38. Valdenmar Publishing Co. (1995).])

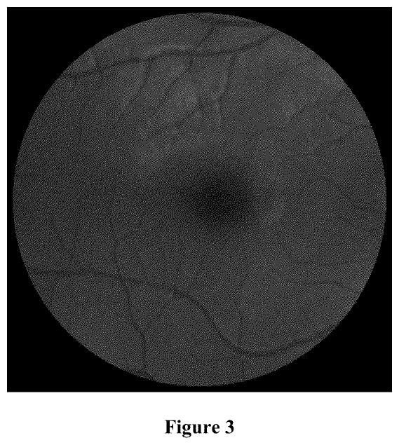

FIG. 3: Autofluorescence image of the macula generated using 488 nm excitation. The light absorbed by macular pigment and lipofuscin generates lipofuscin autofluorescence.

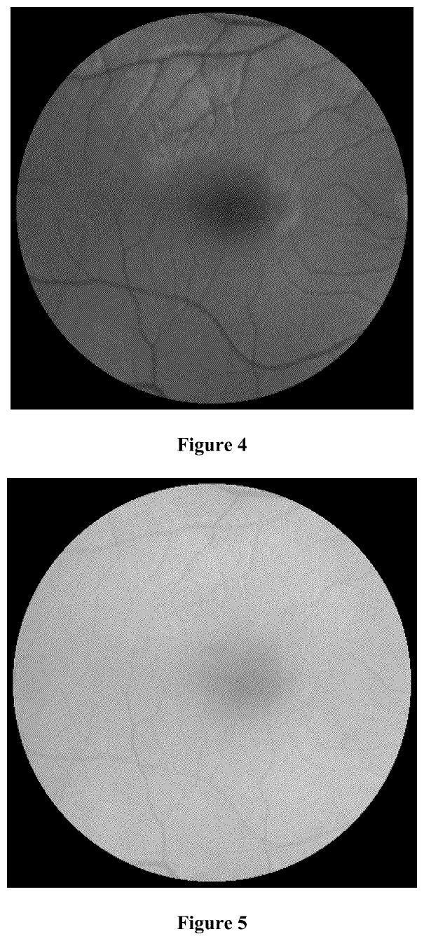

FIG. 4: Autofluorescence image of the macula generated using 514 nm excitation. This wavelength is also absorbed by macular pigment and lipofuscin, generating lipofuscin autofluorescence.

FIG. 5: Autofluorescence image of the macula generated using 600 nm excitation. The light is absorbed by lipofuscin but not by macular pigment, generating lipofuscin autofluorescence and accounting for melanin absorption.

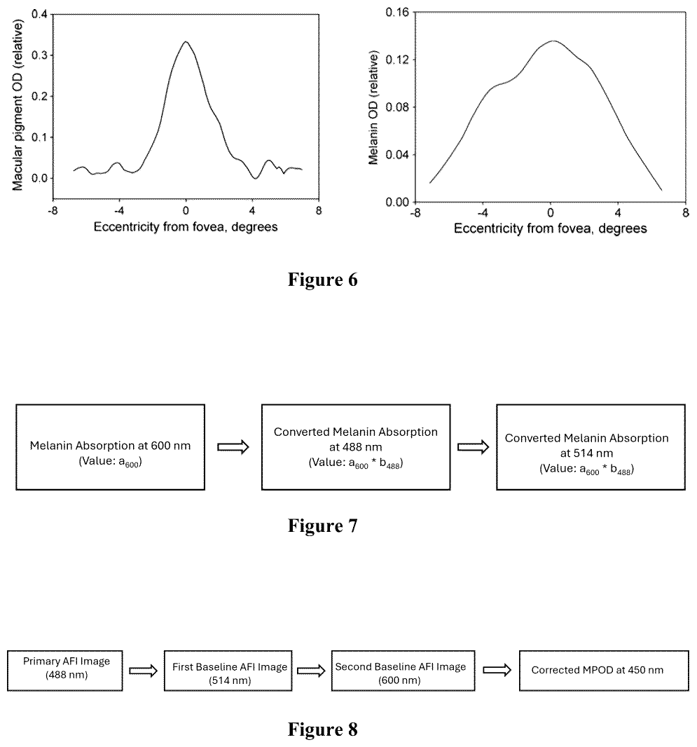

FIG. 6: Graph showing macular pigment optical density (MPOD) and melanin optical density (OD) in one subject, demonstrating that melanin accounts for approximately 30% of the apparent MPOD. This underscores the importance of correcting for melanin absorption to obtain accurate MPOD measurements. (Source: [https://doi.org/10.1016/j.visres.2007.09.002)])

FIG. 7: Conversion of the melanin absorption coefficient from 600 nm to 488 nm and 514 nm. The absorption at 600 nm is first converted to the equivalent absorption at 488 nm using a compensation factor b488, and then converted to the equivalent absorption at 514 nm using a compensation factor b514. This process corrects for melanin absorption, ensuring accurate measurement of macular pigment optical density (MPOD).

FIG. 8: Calculation process for accurate macular pigment optical density (MPOD) measurement using the triple-wavelength approach. The primary AFI image generated at 488 nm is combined with the first baseline AFI image at 514 nm and the second baseline AFI image at 600 nm. The melanin absorption coefficients are converted and subtracted to obtain the corrected MPOD at 450 nm.

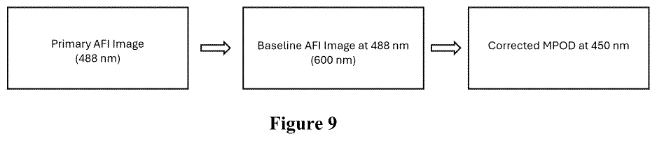

FIG. 9: Calculation process for accurate macular pigment optical density (MPOD) measurement using the dual-wavelength approach. The primary AFI image generated at 488 nm is combined with the baseline AFI image at 600 nm. The melanin absorption coefficient is converted and subtracted to obtain the corrected MPOD at 450 nm.

1- A method for measuring macular pigment optical density using autofluorescence imaging, comprising:
Generating a primary AFI image at a first wavelength absorbed by macular pigment and lipofuscin.
Generating a first baseline AFI image at a second wavelength absorbed by macular pigment and lipofuscin.
Generating a second baseline AFI image at a third wavelength absorbed by lipofuscin but not by macular pigment.
Converting the melanin absorption coefficient from the third wavelength to the first and second wavelengths.
Subtracting the converted melanin values from the primary and first baseline MPOD measurements to find the corrected MPOD.

2- The method of claim 1, wherein the first wavelength is approximately 488 nm, the second wavelength is approximately 514 nm, and the third wavelength is approximately 600 nm.

3- The method of claim 1, wherein the conversion of melanin absorption coefficients includes applying compensation factors for absorption differences between the wavelengths.

4- A method for measuring macular pigment optical density using autofluorescence imaging, comprising:
Generating a primary AFI image at a first wavelength absorbed by macular pigment and lipofuscin.
Generating a baseline AFI image at a second wavelength absorbed by lipofuscin but not by macular pigment.
Converting the melanin absorption coefficient from the second wavelength to the first wavelength.
Subtracting the converted melanin value from the primary MPOD measurement to find the corrected MPOD.

5- The method of claim 4, wherein the first wavelength is approximately 488 nm and the second wavelength is approximately 600 nm.

6- The method of claim 4, wherein the conversion of melanin absorption coefficients includes applying compensation factors for absorption differences between the wavelengths.

7- The method of claim 1 or 4, wherein the corrected MPOD is calculated using a mathematical formulation that accounts for absorption coefficients and logarithmic differences of intensity ratios at the respective wavelengths.

8- A method for measuring macular pigment optical density using autofluorescence imaging, comprising:
Generating a primary AFI image at a first wavelength absorbed by macular pigment and lipofuscin.
Generating a first baseline AFI image at a second wavelength absorbed by macular pigment and lipofuscin.
Generating a second baseline AFI image at a third wavelength absorbed by lipofuscin but not by macular pigment.
Converting the melanin absorption coefficient from the third wavelength to the first and second wavelengths using a mathematical model.
Subtracting the converted melanin values from the primary and first baseline MPOD measurements to find the corrected MPOD using a mathematical formulation.
